SEQUENCE LISTING (25 sequences)

SEQ ID NO: 1 - DNA, 1482 bp, Simmondsia chinensis
  misc_feature: fatty acyl-CoA reductase gene
atggaagagatgggcagcatcctggaatttctggacaataaggctattctggttacaggtgctactggttcactggcgaagatctttgtggaaaaagtgctgcgcagtcaacctaatgttaaaaaactgtatctgctgctgcgcgccaccgatgatgaaactgcagccctgcgtctgcagaatgaagtgtttggcaaagagctgtttaaagttctgaaacagaacctgggggcaaacttctactcgtttgtatcagaaaaagttactgttgtgccaggtgatatcaccggcgaagacctgtgcctgaaagacgtgaatctgaaagaggaaatgtggcgtgaaattgatgtcgtggttaacctggcggcgaccatcaactttattgagcgttatgatgtgtcgctgctgattaacacctatggtgcgaaatatgttctggatttcgctaaaaaatgtaataaactgaaaatctttgttcacgtttcaacggcatacgtttcaggggagaaaaatggtctgattctggagaaaccatactacatgggtgaaagcctgaacgggcgcctgggtctggacattaacgtcgaaaagaaactggtggaggctaaaattaacgaactgcaagcagccggcgccaccgaaaaatccatcaaatcaaccatgaaagacatgggaattgagcgtgcacgccactggggttggccaaatgtgtacgtcttcactaaagcgctgggcgaaatgctgctgatgcaatataaaggggatattccgctgaccattatccgtccgacaattattacgtcaacttttaaggaaccgtttccggggtgggtcgaaggtgttcgcacaattgataacgttccggtgtattacggcaaaggtcgtctgcgctgtatgctgtgcggcccgtctaccattattgatctgatcccggccgatatggtggtgaacgctactattgtcgccatggtggcgcatgcaaaccagcgctatgtggaaccggtgacgtatcacgtgggcagttccgcagccaatccgatgaaactgtcggcgctgcctgaaatggcgcatcgctactttaccaagaatccttggatcaacccggaccgcaacccggtgcatgtgggccgcgcgatggtctttagtagcttttcgacgtttcatctgtatctgacgctgaattttctgctgccgctgaaagtgctggagatcgcgaataccattttctgccagtggtttaaaggtaaatatatggacctgaaacgtaaaacccgcctgctgctgcgcctggtagatatctataaaccatacctgttctttcagggcattttcgacgatatgaacaccgaaaaactgcgcattgcggccaaagaaagcatcgttgaagctgatatgttctattttgacccgcgcgccattaattgggaagattatttcctgaaaacccatttccctggtgtcgtggaacatgtcctgaattga

SEQ ID NO: 2 - DNA, 1851 bp, Arabidopsis thaliana
  misc_feature: at3g11980 gene
atggaggcactctttttgtccagttctagctcctctattgttgcttccaacaagttaactcgcctacacaaccattgtgtgtggagcactgttattagggataaaaagcggtttggtcccacctggtgtcgggtaggcggtgggggtgacggcgggcgaaatagcaatgccgaaaggcccattcgtgtttctagcttattaaaagataggggccaagttctaattcgcgagcaaagcagtccggcgatggatgccgaaactttagtgttgagtccgaatgggaatggtcggaccattgagattaacggcgttaaaaccttgatgcccttctctggagccagcatggtaggtatgaaagaaggtctgggtattatttcctttctccagggaaagaagttcttaattaccgggtctaccgggttcttggcgaaagtgttgatcgaaaaagttctccgtatggcccccgatgtgagtaaaatctatctgttgattaaagcgaaaagtaaggaagcggcgatcgaacgcttgaaaaatgaagtgttggacgctgaattatttaacacactaaaagaaacgcatggcgccagttatatgtcctttatgttaacgaaattgattcctgtgactgggaatatttgtgatagtaatatcgggctccaagctgatagtgctgaagaaattgccaaagaagttgacgtgatcattaacagtgccgccaataccacctttaatgagcgatacgatgttgctctggatattaatacacgaggaccaggaaatttgatggggtttgccaaaaaatgcaagaaattgaaactgtttctgcaagtgagtacggcctatgtaaacggacaacgtcagggtcgtattatggaaaaacccttctctatgggggactgtattgcaaccgagaattttctagaaggcaatcggaaagctttggatgtggatcgtgaaatgaagctggctttagaagccgcccgaaaggggacgcagaatcaagatgaagcacagaaaatgaaagatttaggcctcgaacgggcgcggtcctatggctggcaagatacctacgtattcaccaaggcgatgggcgaaatgatgattaacagcactcgcggcgacgtcccggtggtaattatccgcccatccgtcatcgaatccacctacaaagatccctttcctgggtggatggaaggcaaccgtatgatggatcctattgtgctgtgttatggtaaaggacaattaaccggttttctcgtggaccccaaaggggtgttagacgtagttccggcagatatggtggtgaatgctactctggccgcgatcgcaaaacatggcatggctatgagtgatcccgaacccgaaattaatgtgtatcagatcgccagctccgctattaacccattggtatttgaggacctggccgaactactatacaaccattataaaaccagtccctgcatggattccaagggtgaccccattatggtccgtttaatgaaactcttcaattccgtggatgacttttccgatcatctatggcgcgacgcccaagagcggagtggcctgatgtccggaatgtctagtgtcgatagtaaaatgatgcagaaactgaagtttatctgcaaaaagtccgtcgagcaagccaagcacttggccactatctacgagccttataccttttacggtggccgctttgacaacagtaatacacaacggttgatggaaaatatgtccgaagatgagaagcgagaatttggttttgacgtcggctccattaattggactgattatattacaaacgttcatatccctggactgcgccggcacgtcttaaaaggaagggcctaa

SEQ ID NO: 3 - DNA, 297 bp, Synechocystis sp.
  misc_feature: Promoter fragment Prbc, PCC6803
gcgtcgactcaccatttggacaaaacatcaggaattctaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgacctagatgtctagagcc

SEQ ID NO: 4 - DNA, 198 bp, Synechocystis sp.
  misc_feature: terminator fragment Trbc, PCC6803
cccccggggttacagttttggcaattactaaaaaactgacttcaattcaatgttagcccgctcccgcgggttttttgttgctttttcacagtgactataggtaatcagcaacacaatacggccctgttctttggacagtttttgtataatgttgaccgcatcctgaccggattttttatctaagtggggaagagctcg

SEQ ID NO: 5 - DNA, 440 bp, Synechocystis sp.
  misc_feature: promoter fragment PpetE, PCC6803
aaggattcatagcggttgcccaatctaactcagggagcgacttcagcccacaaaaaacaccactgggcctactgggctattcccattatcatctacattgaagggatagcaagctaatttttatgacggcgatcgccaaaaacaaagaaaattcagcaattaccgtgggtagcaaaaaatccccatctaaagttcagtaaatatagctagaacaaccaagcattttcggcaaagtactattcagatagaacgagaaatgagcttgttctatccgcccggggctgaggctgtataatctacgacgggctgtcaaacattgtgataccatgggcagaagaaaggaaaaacgtccctgatcgcctttttgggcacggagtagggcgttaccccggcccgttcaaccacaagtccctatagatacaatcgccaagaagtatgtc

SEQ ID NO: 6 - DNA, 1681 bp, Synechocystis sp.
  misc_feature: N-terminal sequence of slr0168 gene, PCC6803
ggatcctgggcttcggctatggtggcgtaaagggctggagttagaccaacaaaggccgtggtcagtaaagaaagggaagcggtcagaggacgaatattcggcatagataaattccttgatgtaagtttggtaaagtttttcgatcaaatcaccaaaatcagttccagcacaattattggggtcagttttaaccccggtccagaaaatcggcgataatctcccctgggtcggtttttgactggggaaagaagagaaacacggcctgtgctagtatctaaggcatgaattttcgtgaagttaattataacagtcagtattttgcggctggctaggtgtttaggagtggcaacagcatcatgacgagccatcagctcaacgggcaacggagttaccttcagccgatccgcatcggggtgattggggtgggcaatatgggacagcaccataccagagtcctgagcctgatgaaggatgtggagtttgtgggtattgccgatgtcaatgtggaacggggcctagacaccgccagtaagtatcgggtgcatttttttgaagattatcaggagatgttgccccatgtggatgcggtttgtgtggcggttcccactaggctccatcacgatgtgggcatgaattgtttgcaaaataatgtccatactctgattgaaaaacccattgccgctagcattgctgaagcggaatccctggttaatgccgccgccgatgccaattgcattctccaagtggggcacattgaacgcttcaacccggcatttttagagctaaccaaaattctcaaaacggaagagttattggcgatcgaagcccatcgcatgagtccctattcccagcgggccaatgatgtctccgtggtattggatttgatgatccatgacattgacctgttgctggaattggtgggttcggaagtggttaaactgtccgccagtggcagtcgggcttctgggtcaggatatttggattatgtcaccgctacgttaggcttctcctccggcattgtggccaccctcaccgccagtaaggtcacccatcgtaaaattcgttccatcgccgcccactgcaaaaattccctcaccgaagcggattttctcaataacgaaattttgatccatcgccaaaccaccgctgattggagcgcggactatggccaggtattgtatcgccaggatggtctaatcgaaaaggtttacaccagtaatattgaacctctccacgctgaattagaacattttattcattgtgttaggggaggtgatcaaccctcagtggggggagaacaggccctcaaggccctgaagttagccagtttaattgaagaaatggccctggacagtcaggaatggcatgggggggaagttgtgacagaatatcaagatgccaccctggccctcagtgcgagtgtttaaatcaacttaattaatgcaattattgcgagttcaaactcgataactttgtgaaatattactgttgaattaatctatgactattcaatacacccccctagccgatcgcctgttggcctacctcgccgccgatcgcctaaatctcagcgccaagagtagttccctcaacaccagtattctgctcagcagtgacctattcaatcaggaagggggaattgtaacagccaactatggctttgatggttatatgggaattc

SEQ ID NO: 7 - DNA, 1361 bp, Synechocystis sp.
  misc_feature: C-terminal sequence of slr0168 gene,
ccggtatggatggcaccgatgcggaatcccaacagattgcctttgacaacaatgtggcctggaataacctgggggatttgtccaccaccacccaacgggcctacacttcggctattagcacagacacagtgcagagtgtttatggcgttaatctggaaaaaaacgataacattcccattgtttttgcgtggcccatttttcccaccacccttaatcccacagattttcaggtaatgcttaacacgggggaaattgtcaccccggtgatcgcctctttgattcccaacagtgaatacaacgaacggcaaacggtagtaattacgggcaattttggtaatcgtttaaccccaggcacggagggagcgatttatcccgtttccgtaggcacagtgttggacagtactcctttggaaatggtgggacccaacggcccggtcagtgcggtgggtattaccattgatagtctcaacccctacgtggccggcaatggtcccaaaattgtcgccgctaagttagaccgcttcagtgacctgggggaaggggctcccctctggttagccaccaatcaaaataacagtggcggggatttatatggagaccaagcccaatttcgtttgcgaatttacaccagcgccggtttttcccccgatggcattgccagtttactacccacagaatttgaacggtattttcaactccaagcggaagatattacgggacggacagttatcctaacccaaactggtgttgattatgaaattcccggctttggtctggtgcaggtgttggggctggcggatttggccggggttcaggacagctatgacctgacttacatcgaagatcatgacaactattacgacattatcctcaaaggggacgaagccgcagttcgccaaattaagagggttgctttgccctccgaaggggattattcggcggtttataatcccggtggccccggcaatgatccagagaatggtcccccagggccctttactgtgtccagtagtccccaggtaattaaggtaacggataccatcggccagcccaccaaagtctcctatgtggaagtggatggccccgtattgcgtaatcccttcagtggtactcccattgggcaagaggtgggtttagcggttaaagatctggccacaggtcatgaaatttatcagtacactgacccagatgggaaggtattttatgcttcctttgctgccgctgatgaccaagccacggatttaaccacggcgatcgccaatcccacggccatcgatttaattaacgccaggggatttacggcgggtagttccgtcaccgtatcgggttcctacagtcgggaagccttttttgatggatcc

SEQ ID NO: 8 - DNA, 1948 bp, Unknown
  spectinomycin resistant marker gene Omega
tttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaa

SEQ ID NO: 9 - DNA, 3069 bp, Unknown
  lacZ gene
atgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccacatggggattggtggcgacgactcctggagcccgtcagtatcggcggaattccagctgagcgccggtcgctaccattaccagttggtttggtgtcagaagtaa

SEQ ID NO: 10 - DNA, 21 bp, Artificial Sequence
  Primer alr1524-1
acctccagccattagcgaaac

SEQ ID NO: 11 - DNA, 20 bp, Artificial Sequence
  Primer alr1524-2
ctctcacaattgccctacct

SEQ ID NO: 12 - DNA, 32 bp, Artificial Sequence
  Primer P1
gcgtcgactcaccatttggacaaaacatcagg

SEQ ID NO: 13 - DNA, 36 bp, Artificial Sequence
  Primer P2
gctctagacatctaggtcagtcctccataaacattg

SEQ ID NO: 14 - DNA, 29 bp, Artificial Sequence
  Primer P3
cccccggggttacagttttggcaattact

SEQ ID NO: 15 - DNA, 31 bp, Artificial Sequence
  Primer P4
cgagctcttccccacttagataaaaaatccg

SEQ ID NO: 16 - DNA, 18 bp, Artificial Sequence
  Primer XP-1
agtggttcgcatcctcgg

SEQ ID NO: 17 - DNA, 43 bp, Artificial Sequence
  Primer XP-2
atgaatccttaatcggtaccaaataaaaaaggggacctctagg

SEQ ID NO: 18 - DNA, 43 bp, Artificial Sequence
  Primer XP-3
cccttttttatttggtaccgattaaggattcatagcggttgcc

SEQ ID NO: 19 - DNA, 22 bp, Artificial Sequence
  Primer XP-4
ccagtgaatccgtaatcatggt

SEQ ID NO: 20 - DNA, 22 bp, Artificial Sequence
  lacZ-m1
atggtcaggtcatggatgagca

SEQ ID NO: 21 - DNA, 20 bp, Artificial Sequence
  Primer lacZ-m2
aatccccatgtggaaaccgt

SEQ ID NO: 22 - DNA, 20 bp, Artificial Sequence
  Primer lacZ-m3
acggtttccacatggggatt

SEQ ID NO: 23 - DNA, 24 bp, Artificial Sequence
  Primer M13-Rev
agcggataacaatttcacacagga

SEQ ID NO: 24 - DNA, 30 bp, Artificial Sequence
  Primer far-1
gggtctagaatggaagagatgggcagcatc

SEQ ID NO: 25 - DNA, 33 bp, Artificial Sequence
  Primer far-2
aaacccgggatcaattcaggacatgttccacga

(19) United States
(12) Patent Application Publication
(10) Pub. No.: US 2012/0029248 A1
(43) Pub. Date: Feb. 2, 2012

(54) CONSTRUCTS, VECTORS AND CYANOBACTERIA FOR THE SYNTHESIS OF FATTY ALCOHOLS, AND METHODS FOR PRODUCING FATTY ALCOHOLS IN CYANOBACTERIA

(75) Inventors: Xuefeng LU, Qingdao City (CN); Xiaoming TAN, Qingdao City (CN); Qianqian GAO, Qingdao City (CN); Lun YAO, Qingdao City (CN); Fengxia QI, Qingdao City (CN)

(73) Assignee: SHELL OIL COMPANY, Houston, TX (US)

(21) Appl. No.: 13/170,414

(22) Filed: Jun. 28, 2011

(30) Foreign Application Priority Data
Jun. 30, 2010 (CN) 201010213758.5

(51) Int. Cl. C07C 31/125; C12N 1/13; C07C 11/02; C07C 9/15; C12P 5/02; C12N 15/74; C12P 7/04

(52) U.S. Cl. 568/840; 435/320.1; 435/257.2; 435/157; 585/16; 435/167

(57) ABSTRACT

Constructs, vectors and cyanobacteria for the synthesis of fatty alcohols, and methods for producing fatty alcohols in cyanobacteria is disclosed.

### TECHNICAL FIELD OF THE INVENTION

The present invention relates to a construct for the synthesis of fatty alcohols in cyanobacteria, a vector comprising the construct, a cyanobacterium comprising the construct or transformed by the vector, and a method for producing fatty alcohols in cyanobacteria.

### BACKGROUND OF THE INVENTION

Currently, the sustainable development of economy and society is increasingly restricted by energy and environment related problems. Renewable biofuels are considered as an effective way to solve said problems.

Technical routes for the production of bio-ethanol are relatively well developed. However, ethanol as a fuel has some drawbacks, namely: (1) low energy density; (2) high volatility; (3) problems caused by its high solubility in water, such as the increased toxicity for microorganisms during fermentation, the high cost for the removal of water phase during distillation separation process and the corrosion of pipelines during transportation.

It would be desirable for a biofuel to have properties such as high energy density, low moisture absorption, low volatility, and/or compatibility with existing engines and transport facilities.

Recently, biofuel components prepared from high quality fatty acids, such as long chain fatty alcohols and long chain biologic hydrocarbons are drawing more and more attention.

S. K. Lee et. al. in their article titled “Metabolic engineering of microorganisms for biofuels production: from bugs to synthetic biology to fuels”, published in Current Opinion in Biotechnology, volume 19, issue 6, December 2008 pages 556 to 563 provide a review concerning the status and prospective of such biofuels.

E. J. Steen et. al. in their article titled “Microbial production of fatty-acid-derived fuels and chemicals from plant biomass”, published in Nature, volume 463, 28 Jan. 2010, pages 559 to 562 describe the engineering of Escherichia coli to produce structurally tailored fatty esters (biodiesel), fatty alcohols, and waxes directly from simple sugars.

WO2007/136762 describes the production of fatty acid derivatives by genetically engineered microorganisms such as E. coli and Saccharomyces cerevisiae. It is indicated that the fatty acid derivatives can be useful as biofuels and speciality chemicals.

At present, the microorganism systems used for studying biofuels are primarily heterotrophic microorganisms represented by E. coli and Saccharomyces cerevisiae.

S. A. Angermayr et. al. in their article titled “Energy biotechnology with cyanobacteria”, published in Current Opinion in Biotechnology, volume 20, issue 3, June 2009, pages 257 to 263, describes the possibility to fortify photosynthetic organisms with the ability to produce biofuels. The article describes an approach to redirect cyanobacterial intermediary metabolism by channeling intermediates into fermentative metabolic pathways.

J. Dexter et. al. in their article titled “Metabolic engineering of cyanobacteria for ethanol production”, published in Energy & Environmental Science, volume 2, issue 8, 2009, pages 857 to 864 describe the conversion from solar energy to bioethanol (yield of 5.2 mmol/OD₇₃₀/L/d) by co-expressing the genes of pyruvate decarboxylase and ethanol dehydrogenase derived from Zymomonas mobilis in Synechocystis sp. PCC6803.

Pengcheng Fu, in his article titled “Genome-scale modeling of Synechocystis sp. PCC 6803 and prediction of pathway insertion”, published in the Journal of Chemical Technology & Biotechnology volume 84, issue 4, April 2009, pages 473 to 483, describes a reconstruction of a genome-scale Synechocystis sp. PCC 6803 metabolic network, including 633 genes, 704 metabolites and 831 metabolic reactions. Heterotrophic, photoautotrophic and mixotrophic growth conditions were simulated and the Synechocystis model was used for in silico predictions for the ethanol fermentation pathway.

P. Lindberg et al. in their article titled “Engineering a platform for photosynthetic isoprene production in cyanobacteria, using Synechocystis as the model organism”, published in Metab Eng. volume 12, issue 1, October 2009, pages 70-79 describe the genetic engineering of the cyanobacterium synechocystis, conferring the ability to generate volatile isoprene hydrocarbons from CO(2) and H(2)O. Heterologous expression of the Pueraria montana (kudzu) isoprene synthase (IspS) gene in Synechocystis enabled photosynthetic isoprene generation in these cyanobacteria.

S. Atsumi et al., in their article titled “Direct photosynthetic recycling of carbon dioxide to isobutyraldehyde”, published in Nature Biotechnology, vol 27, pages 1177 to 1180 describes the use of genetically engineered Synechococcus elongatus PCC7942 to produce isobutyraldehyde and isobutanol directly from CO2. Productivity was increased by overexpression of ribulose 1,5-bisphosphate carboxylase/oxygenase (Rubisco).

X. Liu et al., in their article titled “Production and secretion of fatty acids in genetically engineered cyanobacteria.” published in Proceedings of the National Academy of Sciences of the USA, 29 Mar. 2010, describe the production and secretion of free fatty acids in genetically modified Synechocystis sp. PCC6803.

### SUMMARY OF THE INVENTION

The inventors of the present invention, for the first time, successfully produced fatty alcohols in cyanobacteria.

The present invention accordingly provides a construct used for synthesizing fatty alcohols in cyanobacteria, comprising a promoter having activity in cyanobacteria and a fatty acyl-CoA reductase gene under the control of the promoter.

In addition, the present invention provides a vector comprising such a construct; a cyanobacterium comprising the construct or transformed by the vector; and a method for producing fatty alcohols and/or biologic hydrocarbons in a cyanobacterium, comprising culturing a cyanobacterium comprising the construct or transformed by the vector under conditions suitable for the synthesis of fatty alcohols; and extracting the desired fatty alcohols from the obtained culture.

It is an advancement in the art to provide a method for producing fatty alcohols and/or long chain biologic hydrocarbons in a cyanobacterium.

It is further advantageous to construct a route for synthesizing fatty alcohols in cyanobacteria so as to achieve the in vivo synthesis of fatty alcohols in microorganisms.

### BRIEF DESCRIPTION OF THE DRAWINGS

The invention has been illustrated by the non-limiting following figures:

FIG. 1 represents the basic structure of plasmid pFQ9R, in which the Omega fragment of spectinomycin resistance gene, the P_{rbc} promoter and the T_{rbc} terminator are between the upstream and downstream fragments of slr0168 gene of Synechocystis sp. PCC6803; and XbaI and SmaI restriction sites are between the promoter and the terminator.

FIG. 2 represents the basic structure of plasmid pXT14, which is obtained by cloning far gene (far_jojoba) (SEQ ID NO: 1) from Simmondsia chinensis into the plasmid pFQ9R.

FIG. 3 represents the basic structure of plasmid pXT37a, in which the Omega fragment of spectinomycin resistance gene, the P_{petE} promoter and the lacZ gene are between the upstream and downstream fragments of slr0168 gene of Synechocystis sp. PCC6803; and NdeI and EcoRI restriction sites are at the two ends of the lacZ gene.

FIG. 4 represents the basic structure of plasmid pXT37b, which is similar to plasmid pXT37a, except that the insertion direction of the fragment consisting of Omega fragment, P_{petE} promoter and lacZ gene is contrary to that in plasmid pXT37a.

FIG. 5 represents the basic structure of plasmid pXT34, which is obtained by cloning at3g11980 gene (SEQ ID NO: 2) from Arabidopsis thaliana into the plasmid pXT37a, wherein the at3g11980 gene is located downstream of the P_{petE} promoter.

FIG. 6 represents the basic structure of plasmid pXT51, which is obtained by cloning far gene (far_jojoba) (SEQ ID NO: 1) from Simmondsia chinensis into the plasmid pXT37b, wherein the far gene is located downstream of the P_{petE} promoter.

FIG. 7 represents the basic structure of plasmid pLY2, which is obtained by inserting the Omega fragment of spectinomycin resistance gene between the upstream and downstream fragments of slr0168 gene of Synechocystis sp. PCC6803, and cloning the entire construct into the vector pUC9.

FIG. 8 illustrates the production of fatty alcohols in the cells of the genetically engineered strain Syn-LY2 after 8 days of culturing (the determination results of GC-MS), wherein C15-OH represents 1-pentadecanol (used as internal standard), C16-OH represents 1-hexadecanol, and C18-OH represents 1-octadecanol.

FIG. 9 illustrates the production of fatty alcohols in the cells of the genetically engineered strain Syn-XT14 after 8 days of culturing (the determination results of GC-MS), wherein C15-OH represents 1-pentadecanol (used as internal standard), C16-OH represents 1-hexadecanol, and C18-OH represents 1-octadecanol.

FIG. 10 illustrates the production of fatty alcohols in the cells of the genetically engineered strain Syn-XT34 after 8 days of culturing (the determination results of GC-MS), wherein C15-OH represents 1-pentadecanol (used as internal standard), C16-OH represents 1-hexadecanol, and C18-OH represents 1-octadecanol.

FIG. 11 illustrates the production of fatty alcohols in the cells of the genetically engineered strain Syn-XT51 after 8 days of culturing (the determination results of GC-MS), wherein C15-OH represents 1-pentadecanol (used as internal standard), C16-OH represents 1-hexadecanol, and C18-OH represents 1-octadecanol.

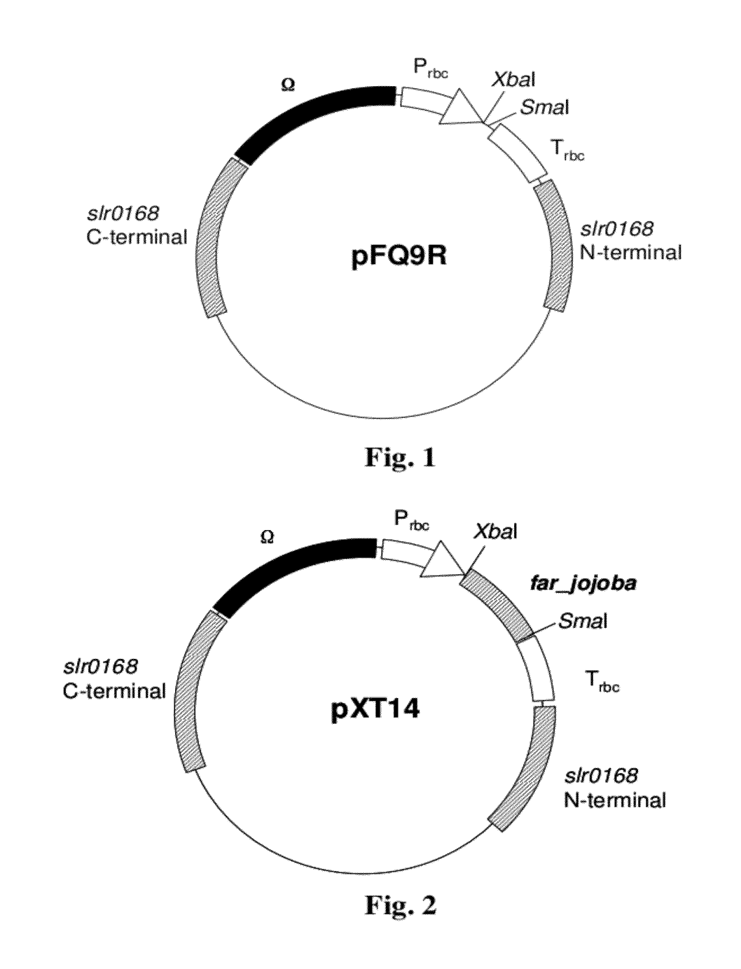

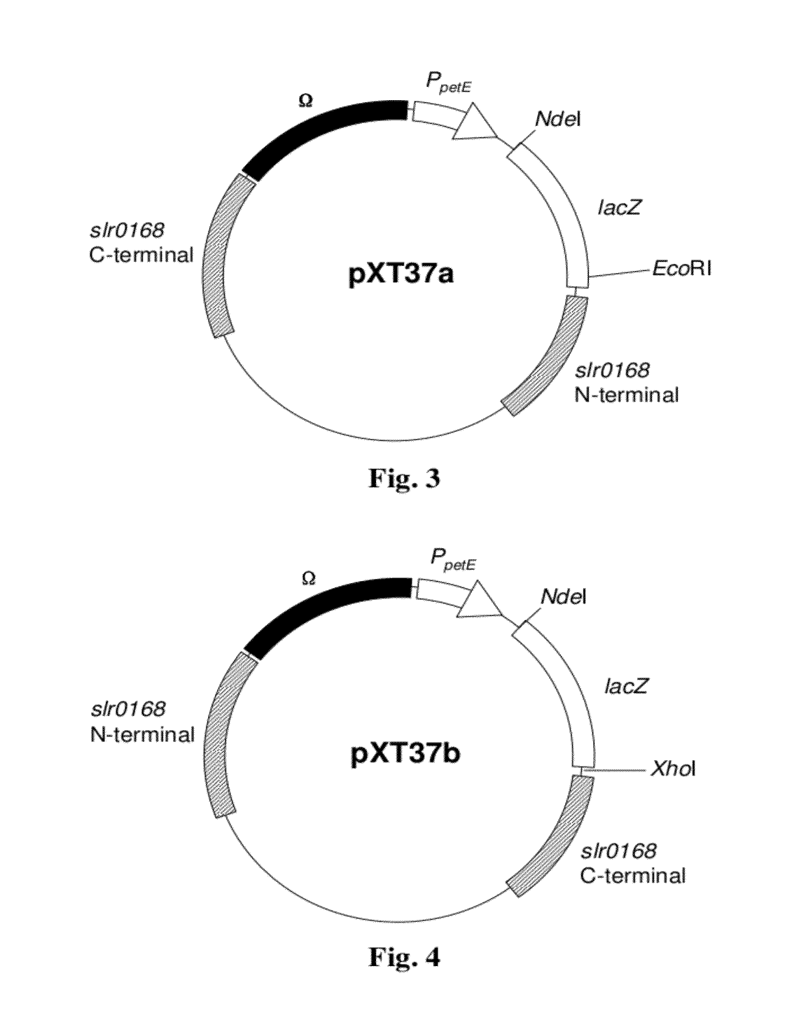

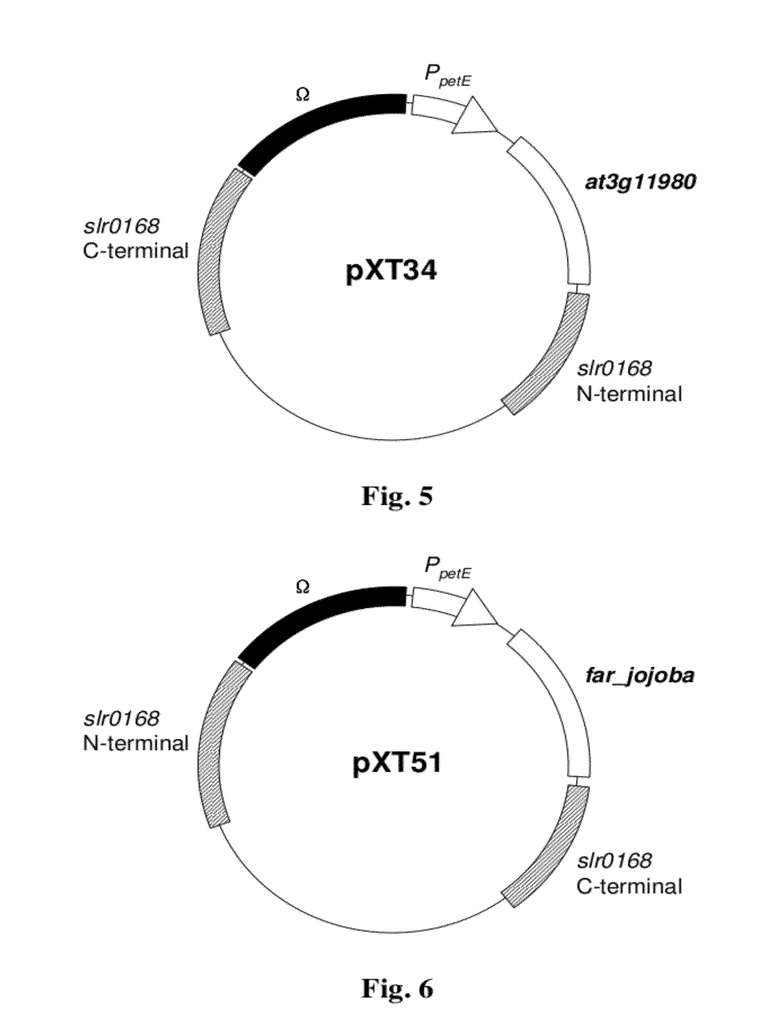

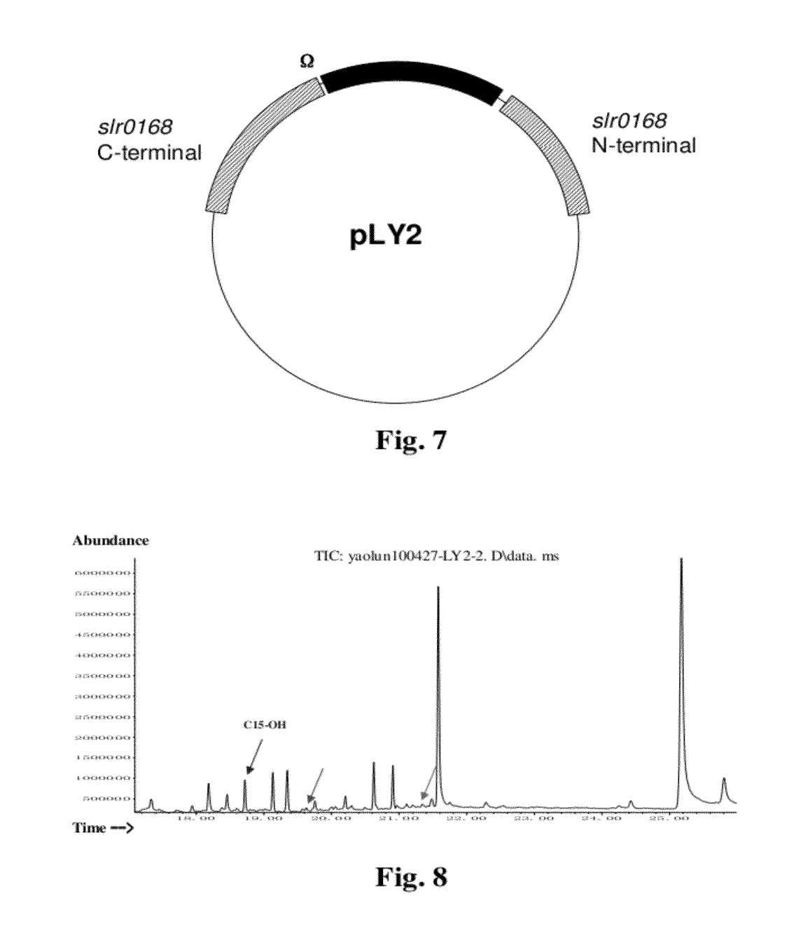

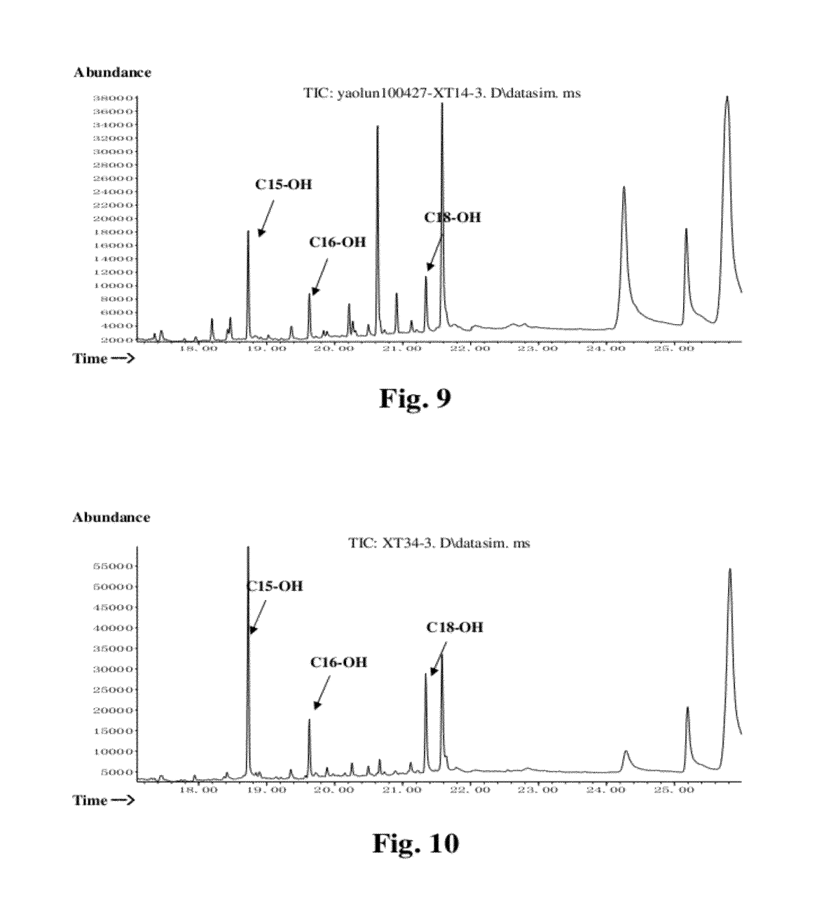

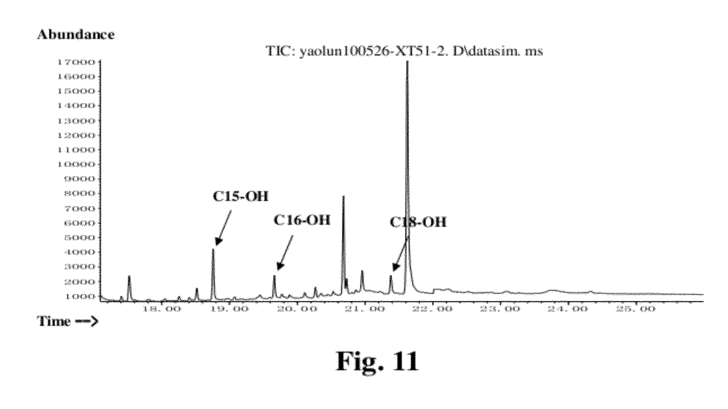

### DESCRIPTION OF THE SEQUENCES

SEQ ID NO: 1: the sequence of fatty acyl-CoA reductase gene from (Simmondsia chinensis) (artificially synthesized gene).

SEQ ID NO: 2: the artificially synthesized sequence according to at3g11980 gene of Arabidopsis thaliana.

SEQ ID NO: 3: the sequence of the promoter fragment P_{rbc} at the upstream of ribulose-1,5-diphosphate carboxylase large-subunit gene rbcL from Synechocystis sp. PCC6803 (NCBI ID: NC_{—}000911).

SEQ ID NO: 4: the sequence of the terminator fragment T_{rbc} at the downstream of ribulose-1,5-diphosphate carboxylase operator from Synechocystis sp. PCC6803 (NCBI ID: NC_{—}000911).

SEQ ID NO: 5: the sequence of the promoter fragment P_{petE} at the upstream of the plastocyanin gene petE from Synechocystis sp. PCC6803 (NCBI ID: NC_{—}000911).

SEQ ID NO: 6: the N-terminal sequence (also comprising a part of the upstream sequence of the gene) of slr0168 gene from Synechocystis sp. PCC6803 (NCBI ID: NC_{—}000911).

SEQ ID NO: 7: the C-terminal sequence (also comprising a part of the downstream sequence of the gene) of slr0168 gene from Synechocystis sp. PCC6803 (NCBI ID: NC_{—}000911).

SEQ ID NO: 8: the sequence of the Omega fragment cloned into the plasmid pRL57 (NCBI ID: L05082).

SEQ ID NO: 9: the sequence of the lacZ gene cloned into the plasmid pHB1567 (NCBI ID: AP009048).

SEQ ID NO: 10: the sequence of the primer alr1524-1.

SEQ ID NO: 11: the sequence of the primer alr1524-2.

SEQ ID NO: 12: the sequence of the primer P1.

SEQ ID NO: 13: the sequence of the primer P2.

SEQ ID NO: 14: the sequence of the primer P3.

SEQ ID NO: 15: the sequence of the primer P4.

SEQ ID NO: 16: the sequence of the primer XP-1.

SEQ ID NO: 17: the sequence of the primer XP-2.

SEQ ID NO: 18: the sequence of the primer XP-3.

SEQ ID NO: 19: the sequence of the primer XP-4.

SEQ ID NO: 20: the sequence of the primer lacZ-m1.

SEQ ID NO: 21: the sequence of the primer lacZ-m2.

SEQ ID NO: 22: the sequence of the primer lacZ-m3.

SEQ ID NO: 23: the sequence of the primer M13-Rev.

SEQ ID NO: 24: the sequence of the primer far-1.

SEQ ID NO: 25: the sequence of the primer far-2.

### DETAILED DESCRIPTION OF THE INVENTION

The following terms will be understood as defined herein unless otherwise stated. Such definitions include without recitation those meanings associated with these terms known to those skilled in the art.

By a “Cyanobacterium” is understood a member from the group of photoautotrophic prokaryotic microorganisms, which can utilize solar energy and fix carbon dioxide. Cyanobacteria are sometimes also referred to as blue-green algae.

By a “Fatty acyl-CoA reductase” is understood an enzyme capable of catalyzing the conversion reaction of fatty acyl-CoA to fatty alcohols.

By a “construct” is herein understood a segment comprising one or more nucleic acids, for example a DNA fragment. The construct is suitably an artificially constructed segment of one or more nucleic acids. The construct can be used to subclone one or more of the nucleic acids, for example a DNA fragment, into a vector.

“Ribulose-1,5-bisphosphate carboxylase/oxygenase” (Rubisco) is an enzyme that catalyzes the first reaction of a so-called Calvin cycle in photosynthesis. It may consist of two subunits and the genes encoding the two subunits can be located in one and the same operator in the Synechocystis sp. PCC6803 genome. In the embodiments of the present invention, a Rubisco promoter (indicated as P_{rbc} in the embodiments of the present invention) may be cloned to drive the expression of fatty acyl-CoA carboxylase gene in cyanobacteria, and the specific sequence for such a Rubisco promoter P_{rbc} is shown in SEQ ID NO: 3.

“Plastocyanin” (PC) is an electron carrier for transferring electron from cytochrome b6/f complex to photosystem I in photosynthesis, and the gene encoding it is abbreviated as “petE”. In the embodiments of the present invention, a petE promoter (indicated as P_{petE} in the embodiments of the present invention) may be cloned to drive the expression of fatty acyl-CoA carboxylase gene in cyanobacteria, and the specific sequence for such a promoter P_{petE} is shown in SEQ ID NO: 5.

A “slr0168 gene” is a gene in the Synechocystis sp. PCC6803 genome, which codes for a protein with unknown function. Previous studies proved that the deletion of this gene does not affect the physiologic activity of cells, so that the site of this gene has been considered as a neutral site in Synechocystis sp. PCC6803 genome. In the embodiments of the present invention a promoter and a fatty acyl-CoA reductase gene may be integrated at this site by homologous recombination so as to express exogenous fatty acyl-CoA reductase in Synechocystis sp. PCC6803.

In the embodiments of the present invention, the term “vector” refers to a self-replicating DNA molecule capable of transferring a DNA fragment (for example the gene of interest) into a recipient cell.

The term “hybridization” is intended to mean the process during which, under suitable conditions, two nucleic sequences bond to one another with stable and specific hydrogen bonds so as to form a double strand. These hydrogen bonds can form between the complementary bases adenine (A) and thymine (T) or uracil (U), which may then be referred to as an A-T bond; or between the complementary bases guanine (G) and cytosine (C), which may then be referred to as a G-C bond. The hybridization of two nucleic sequences may be total (reference is then made to complementary sequences), i.e. the double strand obtained during this hybridization comprises only A-T bonds and C-G bonds. Or the hybridization may be partial (reference is then made to sufficiently complementary sequences), i.e. the double strand obtained comprises A-T bonds and C-G bonds allowing the double strand to form, but also bases not bonded to a complementary base. The hybridization between two complementary sequences or sufficiently complementary sequences depends on the operating conditions that are used, and in particular the stringency. The stringency may be understood to denote the degree of homology; the higher the stringency, the higher percent homology between the sequences. The stringency may be defined in particular by the base composition of the two nucleic sequences, and also by the degree of mismatching between these two nucleic sequences. The stringency can also depend on the reaction parameters, such as the concentration and the type of ionic species present in the hybridization solution, the nature and the concentration of denaturing agents and/or the hybridization temperature. The appropriate conditions can be determined by those skilled in the art.

Conditions for hybridizing nucleic acid sequences to each other can be described as ranging from low to high stringency. Reference herein to hybridization conditions of low stringency includes from at least about 0% v/v to at least about 15% v/v formamide and from at least about 1 M to at least about 2 M salt for hybridization, and from at least about 1 M to at least about 2 M salt for washing conditions. Preferably, the temperature for hybridization conditions of low stringency is from about 25° C., more preferably about 30° C. to about 42° C.

Reference herein to hybridization conditions of medium stringency includes from at least about 16% v/v to at least about 30% v/v formamide and from at least about 0.5 M to at least about 0.9 M salt for hybridization, and from at least about 0.5 M to at least about 0.9 M salt for washing conditions.

Reference herein to hybridization conditions of high stringency includes from at least about 31% v/v to at least about 50% v/v formamide and from at least about 0.01 M to at least about 0.15 M salt for hybridization, and from at least about 0.01 M to at least about 0.15 M salt for washing conditions. In general, washing is carried out at Tₘ=69.3+0.41 (G+C) %, where Tₘ is in degrees Centigrade and (G+C) % refers to the mole percentage of guanine plus cytosine; in line with the article of J. Marmur et al. titled “Determination of the base composition of deoxyribonucleic acid from its thermal denaturation temperature”. published in Journal of Molecular Biology volume 5, issue 1, July 1962, pages 109-118. However, the Tₘ of a duplex DNA may decrease by 1° C. with every increase of 1% in the number of mismatch base pairs in line with the article of W. M. Bonner et al. titled “A Film Detection Method for Tritium-Labelled Proteins and Nucleic Acids in Polyacrylamide Gels”, published in the European Journal of Biochemistry, volume 46, issue 1, 1974, pages 83-88. Formamide is optional in these hybridization conditions. Accordingly, a particularly preferred non-limiting example of a hybridization condition of low stringency is 6×SSC (Standard Sodium Citrate) buffer, 1.0% w/v SDS (Sodium Dodecyl Sulfate) at 25-42° C.; a particularly preferred non-limiting example of a hybridization condition of medium stringency is 2×SSC (Standard Sodium Citrate) buffer, 1.0% w/v SDS (Sodium Dodecyl Sulfate) at a temperature in the range 20° C. to 65° C.; and a particularly preferred non-limiting example of a hybridization conditions of high stringency is 0.1×SSC (Standard Sodium Citrate) buffer, 0.1% w/v SDS (Sodium Dodecyl Sulfate) at a temperature of at least 65° C. An extensive guide to the hybridization of nucleic acids can be found in Tijssen (1993) “Laboratory Techniques in Biochemistry and Molecular Biology—Hybridization with Nucleic Acid Probes”, Part I, Chapter 2 (Elsevier, New York); Ausubel et al., eds. (1995) “Current Protocols in Molecular Biology”, Chapter 2 (Greene Publishing and Wiley-Interscience, New York); and/or Sambrook et al. (1989) “Molecular Cloning: A Laboratory Manual” (2d ed., Cold Spring Harbor Laboratory Press, Plainview, N.Y.).

The term “identity” or “percent identity” refers to the sequence identity between two amino acid sequences or between two nucleic acid sequences. To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). For example, a “percent identity” is calculated by comparing two optimally aligned sequences over the window of comparison, determining the number of positions at which the identical nucleic acid base or the identical amino acid residue occurs in both sequences to yield the number of matched positions, dividing the number of matched positions by the total number of positions in the window of comparison (i.e., the window size), and multiplying the result by 100 to yield the percentage of sequence identity.

Optimal alignment of sequences for comparison can be conducted, for example, by the local homology algorithm of Smith and Waterman (Adv. Appl Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, BLAST P, BLAST N and TFASTA in the Wisconsin Genetics Software Package, Genetics Computer Group, 575 Science Dr., Madison, Wis.), and/or by manual alignment and visual inspection (see, e.g., Ausubel et al, Current Protocols in Molecular Biology (1995 supplement)).

Percent identities involved in the embodiments of the present invention include at least about 60% or at least about 65% or at least about 70% or at least about 75% or at least about 80% or at least about 85% or at least about 90% or above, such as about 95% or about 96% or about 97% or about 98% or about 99%, such as at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The Cyanobacteria (also known as blue-green algae) in this invention preferably comprise a group of prokaryotic microorganisms capable of performing plant type oxygenic photosynthesis.

The use of cyanobacteria may have the following advantages: (1) cyanobacteria are capable of absorbing solar energy and fixing carbon dioxide as carbon source for autotrophic growth, thereby having low cost for culturing; (2) cyanobacteria are ancient microorganisms and have lived on the earth for billions of years, so that they have remarkable adaptability to the environments, and they grow quickly; (3) cyanobacteria are convenient for genetic manipulations, because their genetic background is clear and genomic sequencing of many species of cyanobacteria has been completed which facilitates the genetic engineering of cyanobacteria. Synechocystis sp. PCC6803 is a preferred unicellular cyanobacteria, because for Synechocystis sp. PCC6803 the whole genome sequencing had been completed in 1996.

The embodiments of the present invention employ a promoter having activity in cyanobacteria. This promoter suitably drives the expression of fatty acyl-CoA reductase in cyanobacteria. In this manner the characteristics of cyanobacteria as photosynthetic organism can be utilized to absorb solar energy, fix carbon dioxide and synthesize fatty alcohols as biofuels. One of the advantages of the present invention is that fatty alcohols are synthesized by using solar energy to fix carbon dioxide in the photosynthetic microorganism cyanobacteria, wherein the energy for synthesizing fatty alcohols is solar energy and the carbon source is carbon dioxide. Thus, the production of biofuels utilizing this technology would not be restricted by the lack of raw materials, and the use of such biofuels would not increase carbon emission, i.e., such biofuels are real zero emission biofuels.

In one aspect, the embodiments of the present invention relate to a construct used for synthesizing fatty alcohols in cyanobacteria, which may comprise a promoter having activity in cyanobacteria as well as a fatty acyl-CoA reductase gene under the control of the promoter.

Further, the construct may comprise a marker gene for screening transformants of cyanobacteria, which is located upstream of the promoter having activity in cyanobacteria.

Further, the construct may comprise, at the two termini thereof, the N-terminal and C-terminal sequences of slr0168 gene of Synechocystis sp. PCC6803, for homologous recombination.

In a preferred embodiment, the promoter having activity in cyanobacteria is selected from the group consisting of the P_{rbc} promoter and the P_{petE} promoter.

In a further preferred embodiment, the fatty acyl-CoA reductase gene may be selected from the group consisting of: fatty acyl-CoA reductase (far) gene from Simmondsia chinensis, for example as set forth in SEQ ID NO: 1; and at3g11980 gene from Arabidopsis thaliana, for example as set forth in SEQ ID NO: 2. In addition, the fatty acyl-CoA reductase gene may be far1 gene from mouse (see for example National Center for Biotechnology Information (NCBI) ID: BC007178); codon-optimized far1 gene from mouse; far2 gene from mouse (see for example NCBI ID: BC055759); or at3g56700 gene from Arabidopsis thaliana. Other suitable fatty acyl-CoA reductase genes include: Francci3_{—}2276 from Frankia sp. CcI3 (see for example NCBI ID: NC_{—}007777); KRH_{—}18580 from Kocuria rhizophila DC2201 (see for example NCBI ID: NC_{—}010617); A20C1_{—}04336 from Actinobacterium PHSC20C1 (see for example NCBI ID: NZ_AAOB01000003); HCH_{—}05075 from Hahella chejuensis KCTC 2396 (see for example NCBI ID: NC_{—}007645); Maqu_{—}2220 from Marinobacter aquaeolei VT8 (see for example NCBI ID: NC_{—}008740); and RED65_{—}09889 from Oceanobacter sp. RED65 (see for example NCBI ID: NZ_AAQH01000001). In addition, the embodiments of the present invention may employ the genes having at least 80% identity, preferably at least 85% identity, more preferably at least 90% identity, even more preferably at least 95% identity and most preferably at least 99% identity to the above-mentioned genes and coding for a protein having fatty acyl-CoA reductase activity; or the genes capable of hybridizing with the above-mentioned genes under stringent hybridization conditions, preferably hybridization conditions of high stringency, and coding for a protein having fatty acyl-CoA reductase activity.

In a further preferred embodiment, the marker gene is the Omega fragment of spectinomycin resistance gene, for example as set forth in SEQ ID NO: 8.

In a further preferred embodiment, the cyanobacterium is Synechocystis sp. PCC6803.

In another aspect, the embodiments of the present invention may relate to a vector comprising the construct as defined above. Preferably, the vector is selected from the group consisting of: plasmid pXT14, which was deposited in China General Microbiological Culture Collection Center under Accession Number of CGMCC 3948 on Jun. 28, 2010, in a form in E. coli (Eco-XT14); plasmid pXT34, which was deposited in China General Microbiological Culture Collection Center under Accession Number of CGMCC 3950 on Jun. 28, 2010, in a form in E. coli (Eco-XT34); and plasmid pXT51, which was deposited in China General Microbiological Culture Collection Center under Accession Number of CGMCC 3949 on Jun. 28, 2010, in a form in E. coli (Eco-XT51).

In another aspect, the embodiments of the present invention may relate to a cyanobacterium comprising the construct as defined above, or a cyanobacterium transformed by the vector as defined above. Preferably, the cyanobacterium is selected from the group consisting of: cyanobacterium Syn-XT14, which was deposited in China General Microbiological Culture Collection Center under Accession Number of CGMCC 3894 on Jun. 10, 2010; cyanobacterium Syn-XT34, which was deposited in China General Microbiological Culture Collection Center under Accession Number of CGMCC 3895 on Jun. 10, 2010; and cyanobacterium Syn-XT51, which was deposited in China General Microbiological Culture Collection Center under Accession Number of CGMCC 3896 on Jun. 10, 2010.

In a further aspect, the embodiments of the present invention may relate to a method for producing fatty alcohols in cyanobacteria, comprising: culturing a cyanobacterium comprising the construct as defined above, or a cyanobacterium transformed by the vector as defined above under conditions suitable for the synthesis of fatty alcohols; and extracting the desired fatty alcohols from the obtained culture.

Fatty alcohols, especially long-chain fatty alcohols, such as 1-hexadecanol and 1-octadecanol, were successfully produced in cyanobacteria via the embodiments of the present invention.

If desired, these fatty alcohols may be converted to hydrocarbons by any manner known by the person skilled in the art to be suitable therefore. Such hydrocarbons can include alkanes (such as hexadecane or octadecane) and/or alkenes (such as 1-hexadecene or 1-octadecene).

The fatty alcohols produced via the embodiments of the present invention and/or the hydrocarbons obtained by converting these fatty alcohols can advantageously be used as biofuel components and/or specialty chemicals. Such a biofuel may advantageously have properties such as high energy density, low moisture absorption, low volatility, and/or compatibility with existing engines and transport facilities. In addition such a biofuel may be considered a real zero emission biofuel.

### EXAMPLES

The invention is further illustrated by the following non-limiting examples.

### Example 1

### Construction of Vectors for the Transformation of Cyanobacteria

A summary of the information on the plasmids and strains used for expressing fatty acyl-CoA reductase in Synechocystis sp. PCC6803 is provided in Table 1.

1. Construction of the Plasmid pFQ9R

PCR was performed by using alr1524-1 (5′-ACCTCCAGCCATTAGCG AAAC-3′) and alr1524-2 (5′-CTCTCACAATTGCCCTACCT-3′) as the primer pair and using the genome of Anabaena PCC7120 as the template, and the PCR product was cloned into the vector pMD18-T (Takara, Catalog No.: D101A) to obtain the plasmid pQL1. DraI (Takara, Catalog No.: D1037A) was used to digest the plasmid pRL57 (Cai Y. and Wolk C. (1990) “Use of a conditionally lethal gene in Anabaena sp. strain PCC 7120 to select for double recombinants and to entrap insertion sequences.” J. Bacteriol 172: starting page 3138), and the Omega fragment of about 1.9 kb was recovered. The plasmid pQL1 was digested with PstI (Takara, Catalog No.: D1073A), and blunt-ended with T4 DNA polymerase (Fermentas, Catalog No.: EP0061). The two fragments were ligated to obtain the plasmid pQL4. PCR was performed by using P1 (5′-GCGTCGACTCACCATTTGGAC AAAACATCAGG-3′) and P2 (5′-GCTCTAGACATCTAGGTCAGTCCT CCATAAACATTG-3′) as the primer pair and using the genome of Synechocystis sp. PCC6803 as the template, and the PCR product was cloned into the vector pMD18-T to obtain the plasmid pFQ1; PCR was performed by using P3 (5′-CCCCCGGGGTTACAGTTTTGGCAATTACT-3′) and P4 (5′-CGAGCTCTTCCCCACTTAGATAAAAAATCCG-3′) as the primer pair and using the genome of Synechocystis sp. PCC6803 as the template, and the PCR product was cloned into the vector pMD18-T to obtain the plasmid pFQ2. SalI (Takara, Catalog No.: D1080A) and XbaI (Takara, Catalog No.: D1093A) were used to cut the P_{rbc} fragment from the plasmid pFQ1; XmaI (New England BioLabs, Catalog No.: R0180S) and Sad (Takara, Catalog No.: D1078A) were used to cut the T_{rbc} fragment from the plasmid pFQ2; the P_{rbc} and T_{rbc} fragments were inserted at corresponding site of the plasmid pQL4 to obtain the plasmid pFQ6.

The plasmid pKW1188 (Williams J. G. K. (1988) “Construction of specific mutations in photosystem II photosynthetic reaction center by genetic engineering methods in Synechocystis 6803” Methods in Enzymology 167: pages 766-778) was digested with EcoRI and self-ligated, then blunt-ended with XmaI, and then it was self-ligated to obtain the plasmid pKW1188SL. HindIII (Takara, Catalog No.: D1060A) and EcoRI (Takara, Catalog No.: D1040A) were used to digest the plasmid pFQ6, and the Omega+P_{rbc}+T_{rbc} fragment was recovered; EcoRI was used to digest the plasmid pKW1188SL; and the two fragments were ligated to obtain the plasmid pFQ9R.

2. Construction of the Plasmids pXT37a and pXT37b

The plasmid pHB1567 (Gao Hong, et al, (2007) “Construction of Copper-Induced Gene Expression Platform in Synechocystis sp. PCC6803”, Acta Hydrobiologica Sinica, Vol. 31, No. 2, pages 240-244) was digested with XbaI, the 5.4 kb fragment was recovered and self-ligated to obtain the plasmid pXT24. The plasmid pXT24 was digested with NdeI (Takara, Catalog No.: D1161A), blunt-ended with T4 DNA polymerase and self-ligated; then, it was digested with EcoRI, blunt-ended with T4 DNA polymerase and self-ligated to obtain the plasmid pXT24a. PCR was performed by using the plasmid pHB1536 (GAO Hong, et al, 2007) as the template and using XP-1 (5′-AGTGGTTCGCATCCTCGG-3′) and XP-2 (5′-ATGAATCCTTAAT CGGTACCAAATAAAAAAGGGGACCTCTAGG-3′) as well as XP-3 (5′-CCCTTTTTTATTTGGTACCGATTAAGGATTCATAGCGGTTGCC-3′) and XP-4 (5′-CCAGTGAATCCGTAATCATGGT-3′) as the primer pair, respectively, the PCR product was recovered, and afterwards it was denatured, annealed and extended; then, PCR was performed by using it as the template and using XP-1 and XP-4 as the primer pair, and the PCR product was cloned into the vector pMD18-T to obtain the plasmid pQL17. The plasmid pQL17 was digested with BglII (Takara, Catalog No.: D1021S) and SphI (Takara, Catalog No.: D1180A), and the recovered fragment was ligated to pHB1536 digested with the same enzymes to obtain the plasmid pQL18. The plasmid pQL18 was digested with XbaI, the Omega+P_{petE}+lacZ fragment was recovered and inserted at the same site of the plasmid pXT24a to obtain the plasmid pXT36a. PCR was performed by using the plasmid pHB1567 as the template and using lacZ-m1 (5′-ATGGTCAGGTCATGGATGAGCA-3′) and lacZ-m2 (5′-AATCCCCATGTGGAAACCGT-3′) as well as lacZ-m3 (5′-ACGGTTT CCACATGGGGATT-3′) and M13-Rev (5′-AGCGGATAACAATTTCACAC AGGA-3′) as the primer pair, respectively, the PCR product was recovered, and afterwards it was denatured, annealed and extended; then, PCR was performed by using it as the template and using lacZ-m1 and M13-Rev as the primer pair, and the PCR product was cloned into the vector pMD18-T to obtain the plasmid pXT30. The plasmid pXT30 was digested with EcoRI and EcoRV, and the recovered fragment was ligated to pXT36a digested with the same enzymes to obtain the plasmid pXT37b. The plasmid pXT37b was digested with XbaI, and the two fragments were recovered, self-ligated and screened to obtain the plasmid pXT37a having an insertion direction contrary to that in pXT37b.

3. Construction of the Plasmid pLY2

The plasmid pRL57 was digested with DraI, and the Omega fragment was recovered; the plasmid pKW1188SL was digested with EcoRI, blunt-ended, and the fragment was recovered; the two fragments were ligated to obtain the plasmid pLY2. This plasmid was used as a control plasmid.

4. Construction of the Plasmid pXT14

PCR was performed by using he plasmid pXL66 (a gift from Professor Chaitan Khosla of Stanford University) as the template and using far-1 (5′-GGGTCTAGAATGGAAGAGATGGGCAGCATC-3′) and far-2 (5′-AAA CCCGGGATCAATTCAGGACATGTTCCACGA-3′) as the primer pair, the PCR product was recovered, digested with XbaI and SmaI, and cloned into the same site of the plasmid pFQ9R to obtain the plasmid pXT14.

5. Construction of the Plasmid pXT51

The plasmid pXL66 was digested with NdeI and XhoI, the far gene fragment of Simmondsia chinensis was recovered and inserted into the same site of the plasmid pXT37b to obtain the plasmid pXT51.

6. Construction of the Plasmid pXT34

According to the sequence of SEQ ID No: 2, at3g11980 gene of Arabidopsis thaliana was synthesized and cloned into the plasmid pUC57 (the synthesis was conducted by Sangon Biotech (Shanghai) Co., Ltd) to obtain the plasmid pXT31. The plasmid pHB1567 was digested with EcoRI and XhoI, and the 5.4 kb fragment was recovered; the plasmid pHB1536 was digested with XhoI and NdeI, and the 2.4 kb fragment was recovered; the plasmid pXT31 was digested with NdeI+EcoRI, and the at3g11980 fragment was recovered; these three fragments were ligated to obtain the plasmid pXT34.

### Example 2

### Transformation of Cyanobacteria and Screening of Transformants

1. 10 mL of algae cells in logarithmic growth phase (OD₇₃₀ of about 0.5-1.0) was taken, and centrifuged to collect the cells; the cells were washed twice with fresh BG11 medium, and then resuspended in 1 mL BG11 medium (1.5 g L⁻¹ NaNO₃, 40 mg L⁻¹ K₂HPO₄.3H₂O, 36 mg L⁻¹ CaCl₂.2H₂O, 6 mg L⁻¹ citric acid, 6 mg L⁻¹ ferric ammonium citrate, 1 mg L⁻¹ EDTA disodium salt, 20 mg L⁻¹ NaCO₃, 2.9 mg L⁻¹ H₃BO₃, 1.8 mg L⁻¹ MnCl₂.4H₂O, 0.22 mg L⁻¹ ZnSO₄.7H₂O, 0.39 mg L⁻¹ NaMoO₄.2H₂O, 0.079 mg L⁻¹ CuSO₄.5H₂O and 0.01 mg L⁻¹ CoCl₂.6H₂O).

2. 0.2 mL of cell suspension was placed in a new EP tube, 2-3 μg of the expression plasmid as listed in Table 1 was added, and the resulting mixture was mixed well and incubated at 30° C. under an illumination condition of 30 μE m⁻² s⁻¹ for 5 hours.

3. The mixture of algae cells and DNA was applied onto a nitrocellulose membrane on BG11 plate (without antibiotics) and cultivated at 30° C. under an illumination condition of 30 μE m⁻² s⁻¹ for 24 hours. Then, the nitrocellulose membrane was transferred to a BG11 plate containing 10 μg mL⁻¹ spectinomycin, and further incubated at 30° C. under a condition of 30 μE m⁻² s⁻¹.

4. After about 5-7 days, the transformants were picked out from the plate, and used to streak the fresh BG11 plate (supplemented with 20 μg mL⁻¹ spectinomycin). After the cells were enriched, they are inoculated into a liquid BG11 medium (containing 20 μg mL⁻¹ spectinomycin) for cultivation.

5. After the cells were transferred twice in a liquid medium, the yield of fatty alcohols was measured.

### Example 3

### Production of Fatty Alcohols by the Genetically Engineered Cyanobacteria

1. Experimental Steps:

(1) Culturing method I: shake-flask culturing. A normal 500-mL conical flask with 300 mL of liquid BG11 medium (containing 20 μg mL⁻¹ spectinomycin) was used for inoculation with an initial concentration (OD₇₃₀ of 0.05), and the culturing was performed at 30° C., under an illumination condition of 30 μE m⁻² s⁻¹ and under aeration with air, for 7-8 days.

Culturing method II: column photo-reactor culturing. Normal glass tubes with a height of 575 mm, a diameter of 50 mm and a liquid volume of 500 mL (loading capacity of about 1 L) were used. The initial inoculation concentration was OD₇₃₀ of 0.5, and the culturing was performed at 30° C., under an illumination condition of 100 μE m⁻² s⁻¹ illumination under aeration with air containing 5% CO₂.

(2) 200 mL of medium was taken, algae cells were collected by centrifugation, and resuspended in 10 mL TE (pH8.0) buffer, and then the cells were disrupted via ultrasonication.

(3) 40 μg pentadecanol (as internal standard) was added to the sonicated cells, and an equivalent volume of chloroform:methanol (v/v 2:1) was added, the resulting mixture was mixed well and kept at room temperature for 0.5 hour.

(4) After centrifuging at 3,000 g for 5 minutes, the organic phase was recovered, and dried at 55° C. under blowing with nitrogen gas.

(5) 1 mL n-hexane was added to dissolve the precipitate. After filtration with 0.45 μm filter membrane, GC-MS analysis was performed.

2. Experimental Results:

Hexadecanol and octadecanol were detected in samples of three strains of genetically engineered cyanobacteria: Syn-XT14, Syn-XT34 and Syn-XT51. The total yields of intracellular fatty alcohols under normal shake-flask culturing conditions as shown in Table 2 were calculated by referring to the internal standard (pentadecanol). The results under column photo-reactor culturing conditions also confirmed the ability of the three strains of genetically engineered cyanobacteria for synthesizing fatty alcohols.

The results indicate that the genetically engineered cyanobacteria Syn-XT14, Syn-XT34 and Syn-XT51 were capable of producing fatty alcohols, and this process for producing fatty alcohols can be enlarged in small scale.

TABLE 1
Information on the plasmids and strains used for expressing
fatty acyl-CoA reductase in Synechocystis sp. PCC6803

| | | | | | Resulting | Synechocystis |
|---|---|---|---|---|---|---|
| FAR gene | Source | Cloning method | Vector | Promoter | plasmid | strain |
| far_jojoba | Simmondsia | Gene synthesis | pFQ9R | P_{rbc} | pXT14 | Syn-XT14 |
| | chinensis | | | | | |
| far_jojoba | Simmondsia | Gene synthesis | pXT37b | P_{petE} | pXT51 | Syn-XT51 |
| | chinensis | | | | | |
| at3g11980 | Arabidopsis | Gene synthesis | pKW1188 | P_{petE} | pXT34 | Syn-XT34 |
| | thaliana | | | | | |

TABLE 2
Yields of fatty alcohols of the genetically engineered strains under normal shake-flask culturing conditions

| Synechocystis | | | C16:0 | C18:0 | Total yield | |
|---|---|---|---|---|---|---|
| strains | Genotype | Final OD | (μg OD⁻¹ L⁻¹) | (μg OD⁻¹ L⁻¹) | (μg OD⁻¹ L⁻¹) | (μg L⁻¹) |
| Syn-LY2 | slr0168:: | 3.492 ± 0.351 | N.D | N.D | N.D | N.D |
| | Omega | | | | | |
| Syn-XT14 | slr0168:: | 2.818 ± 0.391 | 4.48 ± 0.55 | 5.24 ± 2.18 | 9.73 ± 2.73 | 27.78 ± 9.60 |
| | (Omega+P_{rbc}+far_jojoba) | | | | | |
| Syn-XT51 | slr0168:: | 2.709 ± 0.317 | 1.64 ± 0.64 | 3.67 ± 0.71 | 5.32 ± 1.16 | 14.66 ± 5.02 |
| | (Omega+P_{petE}+far_jojoba) | | | | | |
| Syn-XT34 | slr0168:: | 3.396 ± 0.766 | 4.87 ± 0.58 | 2.78 ± 1.18 | 7.65 ± 0.67 | 27.73 ± 9.29 |
| | (Omega+P_{petE}+at3g11980) | | | | | |

N.D = Not detectable

TABLE 3
Yields of fatty alcohols of the genetically engineered strains under column photo-reactor culturing conditions

| Synechocystis | | | C16:0 | C18:0 | Total yield | |
|---|---|---|---|---|---|---|
| strains | Genotype | Final OD | (μg OD⁻¹ L⁻¹) | (μg OD⁻¹ L⁻¹) | (μg OD⁻¹ L⁻¹) | (μg L⁻¹) |
| Syn-LY2 | slr0168:: | 12.376 ± 0.177 | N.D | N.D | N.D | N.D |
| | Omega | | | | | |
| Syn-XT14 | slr0168:: | 12.240 ± 0.190 | 4.37 ± 0.32 | 6.85 ± 1.42 | 11.22 ± 1.73 | 137.63 ± 23.26 |
| | (Omega+P_{rbc}+far_jojoba) | | | | | |
| Syn-XT51 | slr0168:: | 13.387 ± 0.386 | 4.53 ± 1.27 | 5.05 ± 0.50 | 9.58 ± 1.57 | 127.90 ± 18.22 |
| | (Omega+P_{petE}+far_jojoba) | | | | | |
| Syn-XT34 | slr0168:: | 13.152 ± 0.386 | 4.84 ± 0.46 | 2.31 ± 0.14 | 7.15 ± 0.60 | 94.16 ± 10.34 |
| | (Omega+P_{petE}+at3g11980) | | | | | |

N.D = Not detectable

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### Deposition Information of the Samples of Biological Materials

| Strains | Accession No. | Deposition date |
|---|---|---|
| Cyanobacteria Syn-XT14 | CGMCC 3894 | Jun. 10, 2010 |
| Cyanobacteria Syn-XT34 | CGMCC 3895 | Jun. 10, 1010 |
| Cyanobacteria Syn-XT51 | CGMCC 3896 | Jun. 10, 2010 |
| E. coli Eco-XT14, containing the | CGMCC 3948 | Jun. 28, 2010 |
| plasmid pXT14 | | |
| E. coli Eco-XT34, containing the | CGMCC 3950 | Jun. 28, 2010 |
| plasmid pXT34 | | |
| E. coli Eco-XT51, containing the | CGMCC 3949 | Jun. 28, 2010 |
| plasmid pXT51 | | |

All of the above strains were deposited in China General Microbiological Culture Collection Center (CGMCC), Institute of Microbiology, Chinese Academy of Sciences, No. 1 West Beichen Road, Chaoyang District, Beijing 100 101, China by QINGDAO INSTITUTE OF BIOENERGY AND BIPROCESS TECHNOLOGY, CHINESE ACADEMY OF SCIENCE, Songling Road No. 189, Qingdao, 266071, P.R. China.

We claim:

1. A construct, useful for synthesizing fatty alcohols in cyanobacteria, comprising a promoter having activity in cyanobacteria and a fatty acyl-CoA reductase gene under the control of the promoter, and further comprising, at the two termini thereof, the N-terminal and C-terminal sequences of slr0168 gene of Synechocystis sp. PCC6803, for homologous recombination.

2. The construct of claim 1 wherein said fatty acyl-CoA reductase gene is a gene selected from the group consisting of: fatty acyl-CoA reductase gene from Simmondsia chinensis comprising a sequence as set forth in SEQ ID NO:1; at3g11980 gene from Arabidopsis thaliana comprising a sequence as set forth in SEQ ID NO:2; variants of these fatty acyl-CoA reductase genes, wherein the variant has at least 80% sequence identity with a sequence set out in SEQ ID NO:1 or SEQ ID NO:2 and encodes for a protein having fatty acyl-CoA reductase activity; and genes capable of hybridizing with the above mentioned genes and coding for a protein having fatty acyl-CoA reductase activity.

3. The construct of claim 1 further comprising an Omega fragment of spectinomycin resistance gene comprising a sequence set forth in SEQ ID NO:8; or a variant thereof that has at least 80% sequence identity with a sequence set out in SEQ ID NO:8.

4. A construct, useful for synthesizing fatty alcohols in cyanobacteria, comprising a promoter having activity in cyanobacteria and a fatty acyl-CoA reductase gene under the control of the promoter, wherein the promoter having activity in cyanobacteria is selected from the group consisting of: a P_{rbc} promoter comprising a sequence set out in SEQ ID NO:3; a P_{petE} promoter comprising a sequence set out in SEQ ID NO:5; and variants of these promoters, wherein the variant has at least 80% sequence identity with a sequence set out in SEQ ID NO:3 or SEQ ID NO:5.

5. The construct of claim 4 wherein said fatty acyl-CoA reductase gene is a gene selected from the group consisting of: fatty acyl-CoA reductase gene from Simmondsia chinensis comprising a sequence as set forth in SEQ ID NO:1; at3g11980 gene from Arabidopsis thaliana comprising a sequence as set forth in SEQ ID NO:2; variants of these fatty acyl-CoA reductase genes, wherein the variant has at least 80% sequence identity with a sequence set out in SEQ ID NO:1 or SEQ ID NO:2 and encodes for a protein having fatty acyl-CoA reductase activity; and genes capable of hybridizing with the above mentioned genes and coding for a protein having fatty acyl-CoA reductase activity.

6. The construct of claim 5 further comprising an Omega fragment of spectinomycin resistance gene comprising a sequence set forth in SEQ ID NO:8; or a variant thereof that has at least 80% sequence identity with a sequence set out in SEQ ID NO:8.

7. The construct of claim 1 wherein said cyanobacterium is Synechocystis sp. PCC6803.

8. The construct of claim 4 wherein said cyanobacterium is Synechocystis sp. PCC6803.

9. A vector comprising a construct of claim 1.

10. The vector of claim 9 which is selected from the group consisting of: plasmid pXT14, which was deposited in China General Microbiological Culture Collection Center under Accession Number of CGMCC 3948 on Jun. 28, 2010; plasmid pXT34, which was deposited in China General Microbiological Culture Collection Center under Accession Number of CGMCC 3950 on Jun. 28, 2010; and plasmid pXT51, which was deposited in China General Microbiological Culture Collection Center under Accession Number of CGMCC 3949 on Jun. 28, 2010.

11. A vector comprising a construct of claim 4.

12. The vector of claim 11 which is selected from the group consisting of: plasmid pXT14, which was deposited in China General Microbiological Culture Collection Center under Accession Number of CGMCC 3948 on Jun. 28, 2010; plasmid pXT34, which was deposited in China General Microbiological Culture Collection Center under Accession Number of CGMCC 3950 on Jun. 28, 2010; and plasmid pXT51, which was deposited in China General Microbiological Culture Collection Center under Accession Number of CGMCC 3949 on Jun. 28, 2010.

13. A cyanobacterium comprising a construct of claim 1.

14. A cyanobacterium comprising a construct of claim 4.

15. A cyanobacterium which is transformed with a vector of claim 9.

16. A cyanobacterium which is transformed with a vector of claim 11.

17. The cyanobacterium of claim 13 which is selected from the group consisting of: cyanobacterium Syn-XT14, which was deposited in China General Microbiological Culture Collection Center under Accession Number of CGMCC 3894 on Jun. 10, 2010; cyanobacterium Syn-XT34, which was deposited in China General Microbiological Culture Collection Center under Accession Number of CGMCC 3895 on Jun. 10, 2010; and cyanobacterium Syn-XT51, which was deposited in China General Microbiological Culture Collection Center under Accession Number of CGMCC 3896 on Jun. 10, 2010.

18. The cyanobacterium of claim 14 which is selected from the group consisting of: cyanobacterium Syn-XT14, which was deposited in China General Microbiological Culture Collection Center under Accession Number of CGMCC 3894 on Jun. 10, 2010; cyanobacterium Syn-XT34, which was deposited in China General Microbiological Culture Collection Center under Accession Number of CGMCC 3895 on Jun. 10, 2010; and cyanobacterium Syn-XT51, which was deposited in China General Microbiological Culture Collection Center under Accession Number of CGMCC 3896 on Jun. 10, 2010.

19. The cyanobacterium of claim 15 which is selected from the group consisting of: cyanobacterium Syn-XT14, which was deposited in China General Microbiological Culture Collection Center under Accession Number of CGMCC 3894 on Jun. 10, 2010; cyanobacterium Syn-XT34, which was deposited in China General Microbiological Culture Collection Center under Accession Number of CGMCC 3895 on Jun. 10, 2010; and cyanobacterium Syn-XT51, which was deposited in China General Microbiological Culture Collection Center under Accession Number of CGMCC 3896 on Jun. 10, 2010.

20. The cyanobacterium of claim 16 which is selected from the group consisting of: cyanobacterium Syn-XT14, which was deposited in China General Microbiological Culture Collection Center under Accession Number of CGMCC 3894 on Jun. 10, 2010; cyanobacterium Syn-XT34, which was deposited in China General Microbiological Culture Collection Center under Accession Number of CGMCC 3895 on Jun. 10, 2010; and cyanobacterium Syn-XT51, which was deposited in China General Microbiological Culture Collection Center under Accession Number of CGMCC 3896 on Jun. 10, 2010.

21. A method for producing fatty alcohols in a cyanobacterium, comprising:
culturing the cyanobacterium of claims 13 under conditions effective for the synthesis of fatty alcohols; and
extracting the desired fatty alcohols from the obtained culture.

22. The method of claim 21 further comprising converting the fatty alcohols to hydrocarbons.

23. A biofuel comprising one or more fatty alcohols produced by the method of claim 21 or comprising one or more hydrocarbons derived from said one or more fatty alcohols.

24. A method for producing fatty alcohols in a cyanobacterium, comprising:
culturing the cyanobacterium of claims 14 under conditions effective for the synthesis of fatty alcohols; and
extracting the desired fatty alcohols from the obtained culture.

25. The method of claim 24 further comprising converting the fatty alcohols to hydrocarbons.

26. A biofuel comprising one or more fatty alcohols produced by the method of claim 24 or comprising one or more hydrocarbons derived from said one or more fatty alcohols.

27. A method for producing fatty alcohols in a cyanobacterium, comprising:
culturing the cyanobacterium of claims 15 under conditions effective for the synthesis of fatty alcohols; and
extracting the desired fatty alcohols from the obtained culture.

28. The method of claim 27 further comprising converting the fatty alcohols to hydrocarbons.

29. A biofuel comprising one or more fatty alcohols produced by the method of claim 27 or comprising one or more hydrocarbons derived from said one or more fatty alcohols.

30. A method for producing fatty alcohols in a cyanobacterium, comprising:
culturing the cyanobacterium of claims 16 under conditions effective for the synthesis of fatty alcohols; and
extracting the desired fatty alcohols from the obtained culture.

31. The method of claim 30 further comprising converting the fatty alcohols to hydrocarbons.

32. A biofuel comprising one or more fatty alcohols produced by the method of claim 30 or comprising one or more hydrocarbons derived from said one or more fatty alcohols.

33. The method of claim 21 wherein the cyanobacterium is selected from the group consisting of: cyanobacterium Syn-XT14, which was deposited in China General Microbiological Culture Collection Center under Accession Number of CGMCC 3894 on Jun. 10, 2010; cyanobacterium Syn-XT34, which was deposited in China General Microbiological Culture Collection Center under Accession Number of CGMCC 3895 on Jun. 10, 2010; and cyanobacterium Syn-XT51, which was deposited in China General Microbiological Culture Collection Center under Accession Number of CGMCC 3896 on Jun. 10, 2010.

34. A biofuel comprising one or more fatty alcohols produced by the method of claim 33 or comprising one or more hydrocarbons derived from said one or more fatty alcohols.
